(19) United States
(12) Patent Application Publication
(10) Pub. No.: US 2015/0018612 A1
(43) Pub. Date: Jan. 15, 2015

(54) MEDICAL DEVICE, MEDICAL SYSTEM, AND PROGRAM

(71) Applicant: Sony Corporation, Tokyo (JP)

(72) Inventors: Akira Tange, Tokyo (JP); Yoichiro Sako, Tokyo (JP); Yuki Koga, Tokyo (JP); Katsumi Ando, Tokyo (JP)

(73) Assignee: Sony Corporation, Tokyo (JP)

(21) Appl. No.: 14/379,804

(22) PCT Filed: Jan. 30, 2013
(86) PCT No.: PCT/JP2013/052004

§ 371 Date: Aug. 20, 2014

(30) Foreign Application Priority Data
Feb. 29, 2012 (JP) 2012-044376

(51) Int. Cl. A61M 31/00; G06F 19/00; A61J 7/00; A61B 1/04; A61M 5/172

(52) U.S. Cl. CPC A61M 31/002; A61B 1/041; A61M 5/1723; A61J 7/0084; G06F 19/3462; A61M 2210/1042; A61M 2205/3324
USPC 600/109; 604/131; 604/66

(57) ABSTRACT

Provided is a medical device including a plurality of storage units each configured to store a different medicine, and a medicine discharge unit configured to selectively discharge at least one of a first medicine and a second medicine, each of which is stored in the plurality of storage units, when the medical device reaches a periphery of a predetermined site of an intracorporeal site of a subject.

### TECHNICAL FIELD

The present disclosure relates to a medical device, a medical system, and a program.

### BACKGROUND ART

In recent years, capsule-type medical devices that are input into the internal body of a subject have been known. Such devices are known to randomly photograph individual sites of the internal body, to collect samples and the like from the internal body, to discharge medicines, and the like.

Particularly, with regard to capsule-type medical devices that discharge medicines, one that discharges a medicine to a desired position (desired lesion) among sites of the internal body has been proposed.

For example, Patent Literature 1 described below has proposed a capsule-type medical device that sparges a medicine by applying a rotating magnetic field from an external rotating magnetic field generator when the capsule-type medical device moves to a desired site of the internal body of a subject.

In addition, Patent Literature 2 described below has proposed another capsule-type medical device which discharges when an extracorporeal device determines whether a capsule has reached the position of a lesion, and transmits a discharge signal to the capsule when it has reached the position of the lesion, and then the capsule-type medical device receives the discharge signal from the extracorporeal device.

### CITATION LIST

### Patent Literature

Patent Literature 1: JP 2003-325438A

Patent Literature 2: JP 2005-334331A

### SUMMARY OF INVENTION

### Technical Problem

Both of the medical devices described above, however, can store only one kind of medicine and do not have the function of selectively discharging one medicine among a plurality of kinds of medicines.

Thus, the present disclosure proposes a novel and improved medical device, medical system, and program that enable at least one medicine among a plurality of medicines to be selectively discharged.

### Solution to Problem

According to the present disclosure, there is provided a medical device including a plurality of storage units each configured to store a different medicine, and a medicine discharge unit configured to selectively discharge at least one of a first medicine and a second medicine, each of which is stored in the plurality of storage units, when the medical device reaches a periphery of a predetermined site of an intracorporeal site of a subject.

According to the present disclosure, there is provided a medical system including a medical device including a plurality of storage units each configured to store a different medicine, and a medicine discharge unit configured to selectively discharge at least one of a first medicine and a second medicine, each of which is stored in the plurality of storage units, and a control device including a position detection unit configured to detect a position of the medical device, and a control unit configured to perform control in a manner that a control signal for causing at least one of the first medicine and the second medicine to be discharged is transmitted to the medical device when the medical device is determined to have reached a periphery of a predetermined site of an intracorporeal site of a subject based on the position detected by the position detection unit.

### Advantageous Effects of Invention

According to the present disclosure described above, at least one medicine among a plurality of medicines can be selectively discharged.

### BRIEF DESCRIPTION OF DRAWINGS

FIG. 1 is a diagram for describing an overview of a medical system according to an embodiment of the present disclosure.

FIG. 2 is a block diagram showing a configuration of a control device according to a first embodiment.

FIG. 3 is an appearance diagram of a capsule-type medical device according to the first embodiment.

FIG. 4 is a diagram showing an internal structure in a state in which a moving body inside a capsule blocks a plurality of storage units according to the first embodiment.

FIG. 5 is a diagram showing an internal structure of main parts of the capsule in a state in which the moving body inside the capsule moves to cause a first storage unit to communicate with the outside according to the first embodiment.

FIG. 6 is a diagram showing an internal structure of the main parts of the capsule in a state in which the moving body inside the capsule further moves to cause a second storage unit to communicate with the outside according to the first embodiment.

FIG. 7 is a flowchart showing a medicine discharge process according to the first embodiment.

FIG. 8 is a block diagram showing a configuration of a control device according to a second embodiment.

FIG. 9 is a flowchart showing a medicine discharge process according to the second embodiment.

FIG. 10 is a diagram showing an example of a medicine sparging site specifying screen displayed on a display unit of the control device according to the second embodiment.

FIG. 11 is a diagram for describing registration of a first specified site on the medicine sparging site specifying screen displayed on the display unit of the control device according to the second embodiment.

FIG. 12 is a diagram for describing registration of a second specified site on the medicine sparging site specifying screen displayed on the display unit of the control device according to the second embodiment.

FIG. 13 is a flowchart showing a medicine discharge process according to a modified example of the second embodiment.

FIG. 14 is a diagram showing an internal structure of main parts of a capsule-type medical device according to a third embodiment.

FIG. 15 is a flowchart showing a medicine discharge process according to the third embodiment.

FIG. 16 is an example showing an internal structure of a capsule-type medical device according to modified example 2.

FIG. 17 is a block diagram showing a configuration of a capsule-type medical device that performs diagnosis of a condition according to a fourth embodiment.

FIG. 18 is a flowchart showing a medicine discharge process in accordance with the diagnosis of a condition according to the fourth embodiment.

FIG. 19 is a block diagram showing a configuration of a capsule-type medical device that performs detection of a condition according to the fourth embodiment.

FIG. 20 is a flowchart showing a medicine discharge process in accordance with the detection of a condition according to the fourth embodiment.

### DESCRIPTION OF EMBODIMENTS

Hereinafter, preferred embodiments of the present invention will be described in detail with reference to the appended drawings. Note that, in this specification and the drawings, elements that have substantially the same function and structure are denoted with the same reference signs, and repeated explanation is omitted.

In addition, description will be provided in the following order.

1. Overview of a medical system according to an embodiment of the present disclosure

2. Embodiments
2-1. First embodiment
   2-1-1. Configuration of a control device
   2-1-2. Structure of a capsule-type medical device
   2-1-3. Medicine discharge process
2-2. Second embodiment
   2-2-1. Configuration of a control device
   2-2-2. Structure of a capsule-type medical device
   2-2-3. Medicine discharge process
   2-2-4. Specification of a medicine sparging site
2-3. Third embodiment
2-4. Fourth embodiment

3. Conclusion

### 1. Overview of a Medical System According to an Embodiment of the Present Disclosure

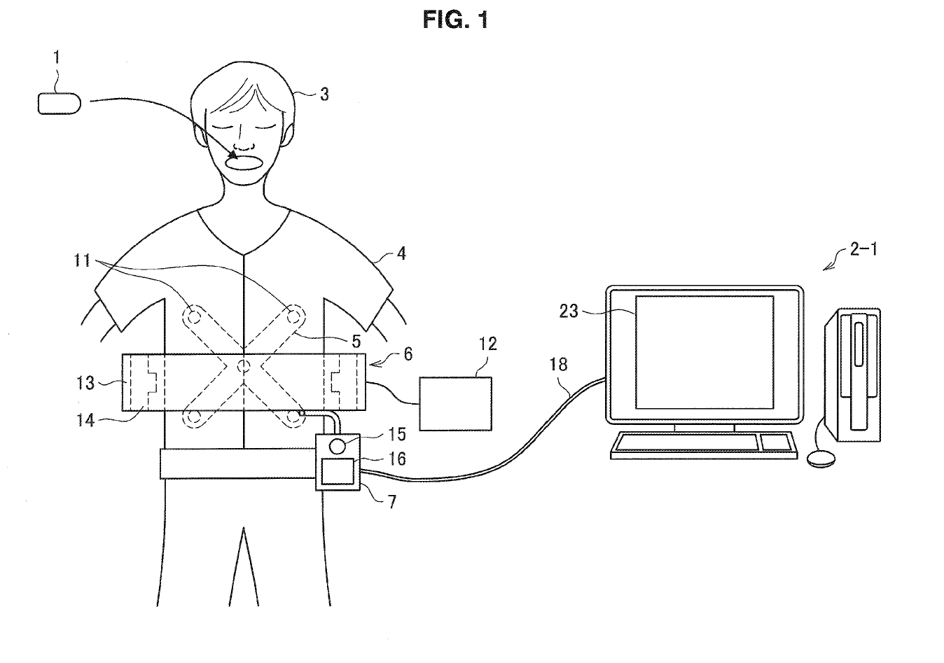

First, an overview of a medical system according to an embodiment of the present disclosure will be described with reference to FIG. 1. As shown in FIG. 1, the medical system according to the embodiment of the present disclosure includes a capsule-type medical device 1 (hereinafter referred to also as a capsule 1), a rotating magnetic field generator 6, and a control device 2-1.

The capsule 1 is ingested orally by a subject 3 as shown in FIG. 1, and wirelessly transmits a signal of an image obtained by optically capturing an internal wall face of a body cavity duct line (captured image) while the capsule passes through the body cavity duct line.

In addition, as shown in FIG. 1, the subject 3 wears a protective shirt 4. The protective shirt 4 has a protection function, and an antenna unit 5 in which a plurality of antennas 11 are installed is mounted thereon. The antenna unit 5 outputs the captured image transmitted from the received capsule 1 and received using the antennas 11 to an extracorporeal unit 7 connected to the antenna unit 5.

The extracorporeal unit 7 is attached to, for example, a belt of the subject 3 with an attachable hook, and holds the captured image output from the antenna unit 5. In addition, the extracorporeal unit 7 is in, for example, a box shape as shown in FIG. 1, and provided with a manipulation button 15 for performing control manipulation and a liquid crystal monitor 16 performing image display on its front face.

In addition, the captured image held in the extracorporeal unit 7 may be displayed on the liquid crystal monitor 16 during or after inspection, or may be transmitted to the control device 2-1 during or after the inspection and then displayed on a display unit 23 of the control device 2-1. The extracorporeal unit 7 and the control device 2-1 may be detachably connected to each by wire other using a communication cable such as a USB cable 18 or the like as shown in FIG. 1, or may be wirelessly connected.

Accordingly, a medical staff can check the captured image of the inside of the body cavity duct line of the subject 3 during or after the inspection from the liquid crystal monitor 16 of the extracorporeal unit 7 or the display unit 23 of the control device 2-1.

Furthermore, as shown in FIG. 1, the rotating magnetic field generator 6 is disposed around the waist or the like of the subject 3. The rotating magnetic field generator 6 is configured such that the magnetic poles of electromagnets 14 are disposed to face each other in a plurality of positions in the circumferential direction of the ring-shaped frame member 13 and includes a drive circuit 12 that supplies a drive signal to the electromagnets 14.

In addition, the capsule 1 of the present embodiment has a structure in which a medicine is stored therein and the medicine is discharged due to generation of a rotating magnetic field. The rotating magnetic field is generated when the drive circuit 12 described above is operated and direct currents are sequentially supplied from the drive circuit 12 to the electromagnets 14 of the plurality of positions as drive signals.

An operation timing of the drive circuit 12 may be based on manipulation of a switch (not shown) of the drive circuit 12 performed by a medical staff who has checked the lesion through the captured image displayed on, for example, the liquid crystal monitor 16 of the extracorporeal unit 7 or the display unit 23 of the control device 2-1. In addition, the drive circuit 12 may be operated according to an actuation signal from the control device 2-1.

Here, when only one kind of medicine is stored in the capsule 1, it is difficult to sparge different medicines to a plurality of lesions, to sparge a plurality of medicines in a time differential manner, or to select an appropriate medicine according to a state of a lesion.

Thus, according to an embodiment of the present disclosure, it is possible to provide a capsule-type medical device that can store a plurality of medicines therein and selectively discharge at least one of the plurality of medicines.

Hereinabove, the overview of the medical system according to the embodiment of the present disclosure has been described. Next, the medical system of the present disclosure will be described in detail exemplifying a plurality of embodiments.

### 2. Embodiments

### 2-1. First Embodiment

A medical system according to a first embodiment includes a capsule-type medical device 1 (capsule 1) input to the internal body of the subject 3 and the control device 2-1 as shown in FIG. 1. Hereinbelow, a basic configuration of the control device 2-1, a structure of the capsule 1, and a medicine discharge process according to the first embodiment will be described in order.

### 2-1-1. Configuration of a Control Device

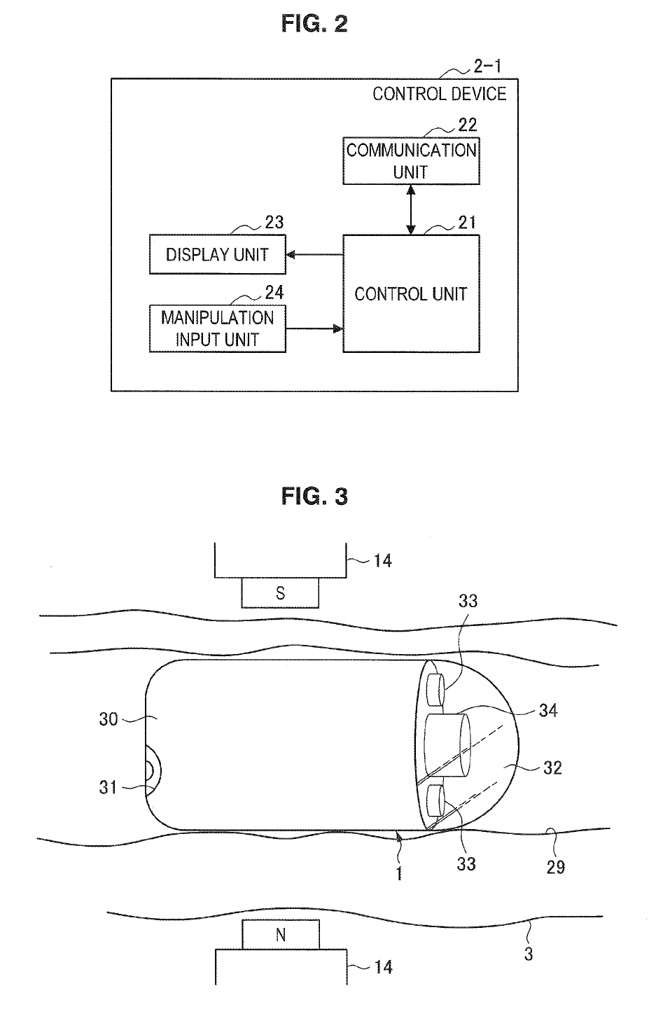

FIG. 2 is a block diagram showing a configuration of the control device 2-1 according to the first embodiment. As shown in FIG. 2, the control device 2-1 has a control unit 21, a communication unit 22, the display unit 23, and a manipulation input unit 24.

The communication unit 22 has a function of being connected to an external device to perform transmission and reception of data. For example, the communication unit 22 is connected to the extracorporeal unit 7 to receive captured images from the extracorporeal unit 7. In addition, the communication unit 22 may be connected to the rotating magnetic field generator 6 in a wired or wireless manner and transmit an actuation signal for operating the drive circuit 12.

The display unit 23 has a function of displaying a screen including an image or text according to control of the control unit 21. In addition, the display unit 23 is realized by an LCD (Liquid Crystal Display), an OLED (Organic Light-Emitting Diode), a CRT (Cathode Ray Tube), or the like.

To be more specific, the display unit 23 displays, for example, the captured image received from the extracorporeal unit 7. Accordingly, the medical staff can check the captured image of the internal body of the subject 3, recognize the position of the capsule 1, and determine whether or not a site is one to be treated with discharge of a medicine. In addition, the display unit 23 may display a manipulation screen for accepting medicine discharge manipulation with respect to the site to be treated inside the body.

The manipulation input unit 24 has a function of detecting manipulation performed by a medical staff and outputting an input signal generated based on the detected manipulation input to the control unit 21. In addition, the manipulation input unit 24 is realized by a mouse, a keyboard, a touch panel, and the like. A medical staff can perform various kinds of manipulation such as the medicine discharge manipulation by manipulating the manipulation input unit 24.

The control unit 21 has a function of controlling the entire control device 2-1. To be more specific, the control unit 21 controls such that, for example, the captured image received by the communication unit 22 is displayed on the display unit 23. In addition, the control unit 21 may control the communication unit 22 to transmit an actuation signal to the rotating magnetic field generator 6 according to the input signal of the medicine discharge manipulation output from the manipulation input unit 24.

Hereinabove, the configuration of the control device 2-1 according to the first embodiment has been described in detail. Next, a structure of the capsule-type medical device 1 according to the first embodiment will be described with reference to FIGS. 3 to 6.

### 2-1-2. Structure of a Capsule-Type Medical Device

FIG. 3 is an appearance diagram of the capsule-type medical device 1 according to the first embodiment. The capsule 1 inserted into a body cavity duct line 29 of the subject 3 as shown in FIG. 3 is in a substantially cylindrical shape, and is covered with an external case 30 that shields the rear end of the capsule 1 in round shapes. In addition, an opening 31 through which a medicine is discharged is provided in a rear part of the external case 30, and a transparent cover 32 in a hemispheric shape is water-tightly connected and fixed to a tip end part of the external case 30.

In a hermetically sealed container inside the transparent cover 32, an imaging optical system 34 is provided at the center thereof so as to face the transparent cover 32, and lighting units of white LEDs 33 or the like are disposed in the periphery of the imaging optical system 34 as shown in FIG. 3.

In addition, when the capsule 1 reaches the periphery of a predetermined site inside the body, a rotating magnetic field is generated when a drive current flows in the plurality of electromagnets 14 of the rotating magnetic field generator 6 disposed near the site, and then a medicine is discharged from the opening 31 of the capsule 1.

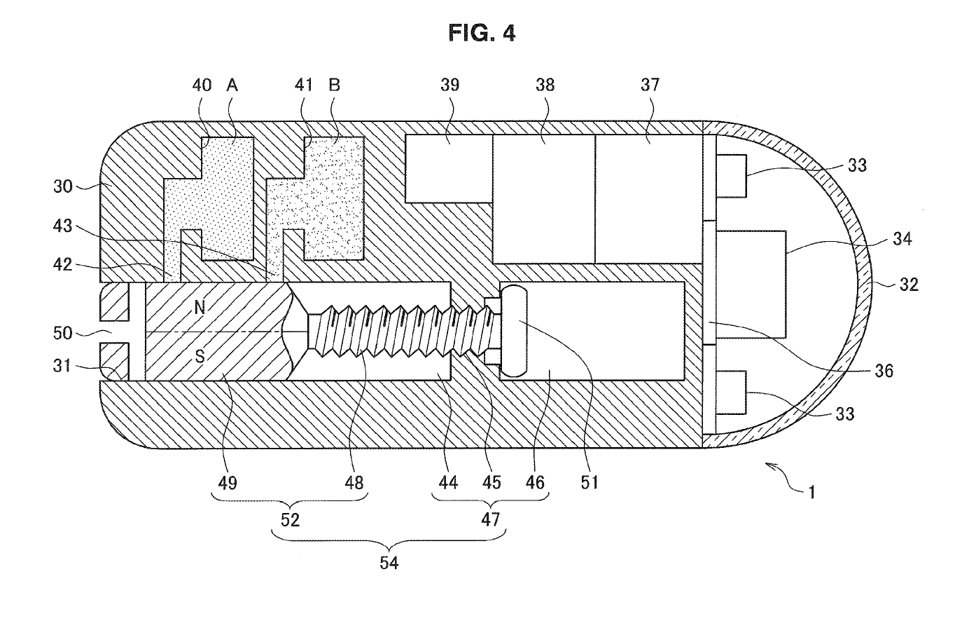

To be more specific, when a moving body 52 rotation-freely provided inside the capsule 1 moves due to the rotating magnetic field, storage units inside the capsule 1 turn from a blocked state to a communicating state with the outside, and then the medicine stored in the storage units is discharged. Hereinbelow, details of such an internal structure of the capsule 1 will be described with reference to FIGS. 4 to 6.

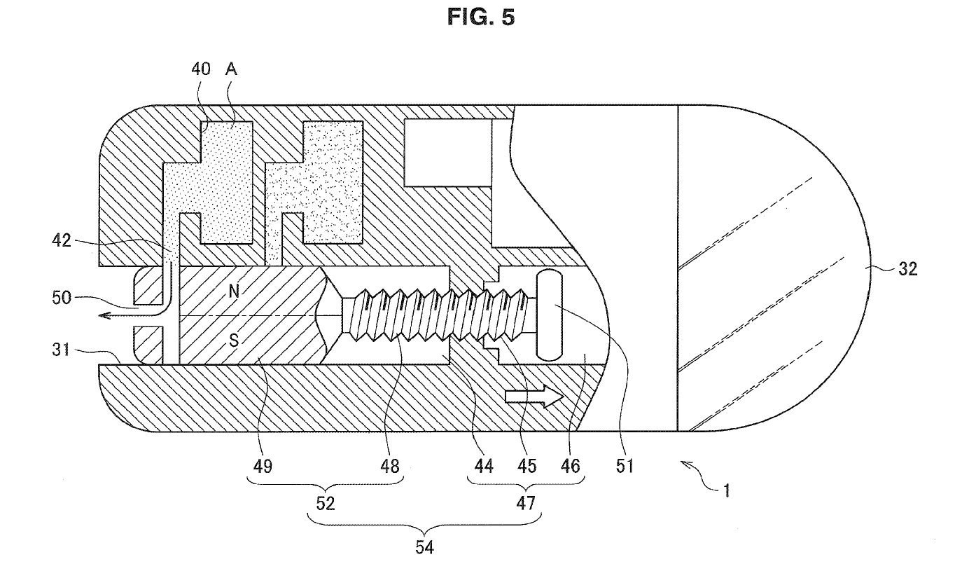

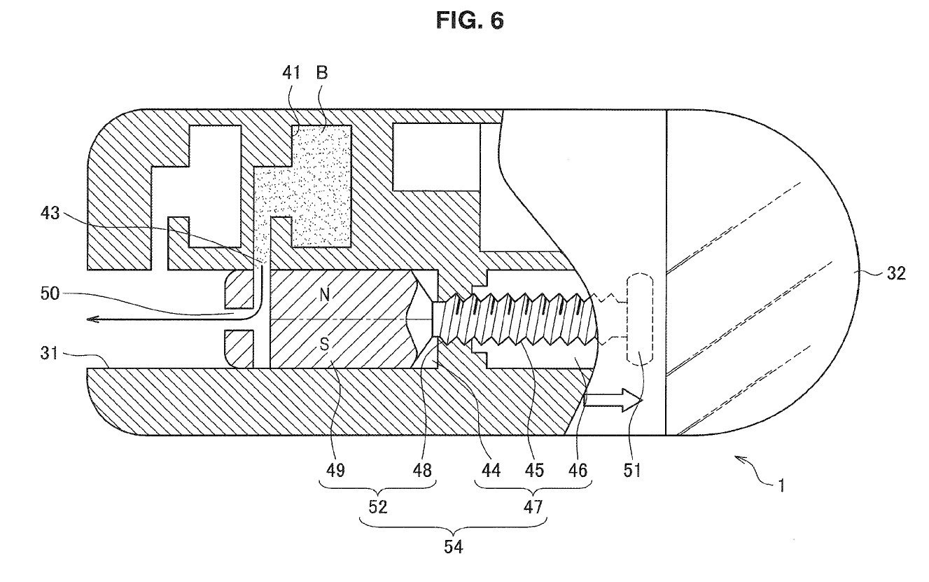

FIG. 4 is a diagram showing the internal structure in a state in which the moving body inside the capsule 1 blocks a plurality of storage units. FIG. 5 is a diagram showing an internal structure of main parts of the capsule 1 in a state in which the moving body inside the capsule 1 moves to cause a first storage unit to communicate with the outside. FIG. 6 is a diagram showing an internal structure of the main parts of the capsule 1 in a state in which the moving body inside the capsule 1 further moves to cause a second storage unit to communicate with the outside.

As shown in FIG. 4, in the image formation position of the imaging optical system 34 disposed at the center of the transparent cover 32 to face each other, an image sensor 36 such as a CMOS imager (or CCD) is disposed.

A control unit 37, a memory and communication unit 38, and a battery 39 are disposed in a rear upper part of the image sensor 36. The control unit 37 drives the image sensor 36, performs signal processing on an output signal of the image sensor 36, or controls other circuits such as the memory and communication unit 38 that will be described next.

The memory and communication unit 38 has functions of memorizing captured image signals (captured images), and of communication of wirelessly transmitting image signals or the like.

The battery 39 is, for example, a button type, is conductive with a wiring substrate that is not illustrated, and is electrically connected to the memory and communication unit 38 via the wiring substrate.

In addition, as shown in FIG. 4, the storage units 40 and 41 are provided in a part compartmented from the battery 39, the memory and communication unit 38, and the control unit 37 by a wall portion of the external case 30 on the rear (left) side of the battery 39.

Medicines each stored in the storage units 40 and 41 are inserted in advance together with a pressurized gas from a lateral hole that is not illustrated. In addition, the lateral hole is sealed with a rubber plug or the like after the insertion of the medicines.

As shown in FIG. 4, while the storage units 40 and 41 are provided to be eccentric on the upper side from the central axis of the capsule 1, a medicine discharge unit 54 that selectively discharges a medicine from each of the storage units is provided on the opposite side of the storage units 40 and 41 to be eccentric on the lower side from the central axis of the capsule 1.

The medicine discharge unit 54 according to the first embodiment is realized by the moving body 52 and a moving body storage unit 47 that supports and has the moving body 52 disposed therein such that the moving body can rotatably move in the longitudinal direction of the capsule 1 as shown in FIG. 4.

The moving body storage unit 47 includes a first recess portion 44 having the opening 31 in its rear end, a screw hole (female screw) 45 formed on the front end side of the first recess portion 44, and a second recess portion 46 communicating with the first recess portion 44 via the screw hole 45. Note that, on a side part of the first recess portion 44, openings 42 and 43 of duct lines each communicating with the storage units 40 and 41 are open.

In addition, the moving body storage unit 47 store and supports the moving body 52 in the state in which a screw portion (male screw portion) 48 provided on the front end side of the moving body 52 is screwed into the screw hole 45.

As described above, the screw portion 48 is provided on the front end of the moving body 52, and a stopper 51 in, for example, a disc shape is provided at the tip end of the screw portion 48 (the front end of the moving body 52). The stopper 51 is stored in the second recess portion 46. In addition, a cylinder portion 49 that fits into the first recess portion 44 is provided on the rear end side of the moving body 52, and in the periphery of the rear end of the cylinder portion 49, a hole 50 in, for example, a T shape is provided.

In the state shown in FIG. 4, the moving body 52 seals the openings 42 and 43 using the cylinder portion 49. Here, the moving body 52 is formed of a permanent magnet of which sides of the central axis indicated by, for example, the dotted chain line (for example, the upper half and the lower half of the central axis) are magnetized to be N and S. Thus, in the state shown in FIG. 4, when a rotating magnetic field is generated by the electromagnets 14 shown in FIG. 3, the moving body 52 is rotated and moves in the tip end direction (to the right) as shown in FIG. 5.

In the state shown in FIG. 5, the T-shaped hole 50 of the moving body 52 communicates with the opening 42, and a first medicine (hereinafter referred to as a medicine A) stored in the storage unit 40 is discharged to the outside of the capsule 1 passing through the T-shaped hole 50 from the opening 42.

In addition, when the moving body 52 is further rotated and moves in the tip end direction (to the right) to the position in which, for example, the stopper 51 reaches a wall face of the second recess portion 46, the T-shaped hole 50 of the moving body 52 communicates with the opening 42 as shown in FIG. 6. Accordingly, a second medicine (hereinafter referred to as a medicine B) stored in the storage unit 41 is discharged to the outside of the capsule 1 passing through the T-shaped hole 50 from the opening 43.

As described above with reference to FIGS. 4 to 6, in the present embodiment, when the moving body 52 included in the medicine discharge unit 54 inside the capsule 1 is rotated and moves according to the generation of the external rotating magnetic field, the openings 42 and 43 communicating with the storage units 40 and 41 in which the medicines are stored are opened or closed.

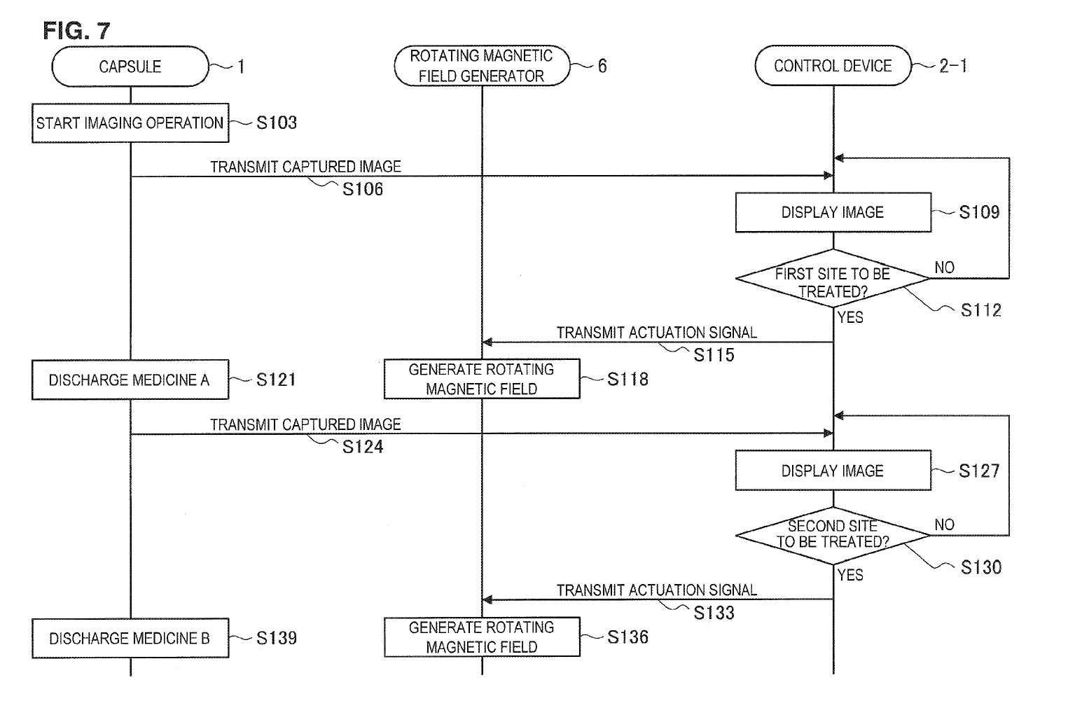

Next, a medicine discharge process performed by the medical system that includes the capsule 1 and the control device 2-1 with the above-described structure will be described in detail with reference to FIG. 7.

### 2-1-3. Medicine Discharge Process

FIG. 7 is a flowchart showing the medicine discharge process according to the first embodiment. As shown in FIG. 7, first, power is input and the capsule 1 ingested by the subject 3 starts an operation of imaging the inside of the body cavity in Step S103.

Next, in Step S106, the memory and communication unit 38 of the capsule 1 transmits the captured image of the inside of the body cavity captured by the image sensor 36 while passing through the body cavity duct line 29 to the control device 2-1 via the extracorporeal unit 7.

Next, in Step S109, the control device 2-1 causes the received captured image to be displayed on the display unit 23. As described above, a medical staff can determine a position of the capsule 1 and a site to be treated with discharge of a medicine based on the captured image displayed on the display unit 23. For example, when the medical staff determines (a periphery of) a first treatment site to which the medicine A (first medicine) is to be discharged based on the captured image, he or she performs medicine discharge manipulation using the manipulation input unit 24.

Next, when the control device 2-1 detects the manipulation performed by the medical staff to discharge the medicine A (manipulation indicating the first treatment site) in Step S112, the control device then transmits an actuation signal to the rotating magnetic field generator 6 in Step S115.

Next, in Step S118, when the drive circuit 12 is operated according to the actuation signal, the rotating magnetic field generator 6 generates a rotating magnetic field while direct currents are sequentially supplied from the drive circuit 12 to the electromagnets 14 in the plurality of positions as drive signals.

Note that a trigger that causes the rotating magnetic field generator 6 to generate a rotating magnetic field is not limited to reception of the actuation signal shown in Step S115 described above. For example, the rotating magnetic field generator 6 may generate a rotating magnetic field having, as a trigger, turning-on of the switch (not illustrated) of the drive circuit 12 by the medical staff who has determined (a periphery of) a site to which the medicine A is to be discharged based on a displayed captured image.

Next, in Step S121, the capsule 1 discharges the medicine A. As described above, when the rotating electric field is applied, the moving body 52 of the capsule 1 is rotated and moves, and thereby the opening 42 of the storage unit 40 in which the medicine A is stored is in the state of communication with the outside via the T-shaped hole 50 of the moving body 52 as shown in FIG. 5. Accordingly, the medicine A of the storage unit 40 is discharged to the outside of the capsule 1 and thereby the medicine A is sparged in the periphery of the first treatment site.

Next, in Steps S124 to S139, the same processes as Steps S106 to S121 are performed, and thus the medicine B (second medicine) is discharged in the periphery of a second treatment site.

In other words, after the discharge process of the medicine A shown in Step S121, the capsule 1 continues to transmit captured images of the inside of the body cavity obtained by imaging of the image sensor 36 while passing through the inside of the body cavity duct line 29 to the control device 2-1 in Step S124.

Next, in Step S127, the control device 2-1 causes the received captured image to be displayed on the display unit 23. Next, in Step S130, when the manipulation performed by the medical staff to discharge the medicine B (manipulation to indicate a second treatment site) is detected, the control device then transmits an actuation signal to the rotating magnetic field generator 6 in Step S133 as in Step S115 described above.

Next, in Step S136, when the drive circuit 12 is operated according to the actuation signal, the rotating magnetic field generator 6 generates a rotating magnetic field while direct currents are sequentially supplied from the drive circuit 12 to the electromagnets 14 in the plurality of positions as drive signals as in Step S118.

Next, In Step S139, the capsule 1 discharges the medicine B. As described above, when the rotating magnetic field is applied, the moving body 52 of the capsule 1 is rotated and moves, and the opening 43 of the storage unit 41 in which the medicine B is stored is in a communicating state with the outside via the T-shaped hole 50 of the moving body 52 as shown in FIG. 6. Accordingly, the medicine B of the storage unit 41 is discharged to the outside of the capsule 1, and thereby the medicine B is sparged in the periphery of the second treatment site.

Then, the capsule 1 emitted to the outside of the body is collected or the like by the medical staff, and thereby treatment using the capsule 1 ends.

As described above, in the medicine discharge process according to the first embodiment, by performing manipulation to discharge the medicines to arbitrary treatment sites while the medical staff checks the captured images, the rotating magnetic fields are generated by the rotating magnetic field generator 6, and then the capsule 1 sequentially discharges the first and second medicines.

### 2-2. Second Embodiment

In the first embodiment described above, the rotating magnetic fields are generated when the medical staff determines peripheries of arbitrary treatment sites (peripheries of the first and second treatment sites) by checking the captured images and then manipulates the manipulation input unit 24 of the control device 2-1 or the switch of the drive circuit 12 at an arbitrary timing.

The medical system of the present disclosure, however, is not limited to the first embodiment, and may be a medical system that can automatically determine, for example, whether or not the capsule 1 has reached a periphery of a pre-specified site. Hereinbelow, a second embodiment that realizes such a medical system will be described in detail.

### 2-2-1. Configuration of a Control Device

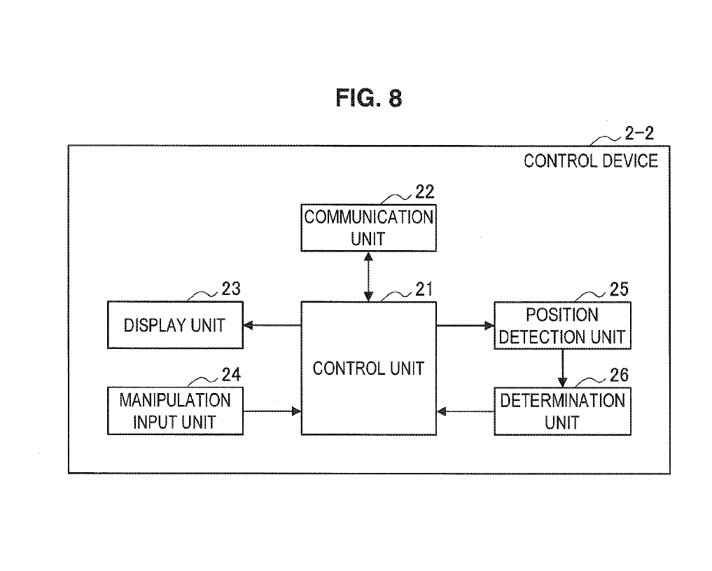

FIG. 8 is a block diagram showing a configuration of a control device 2-2 according to the second embodiment. As shown in FIG. 8, the control device 2-2 has a position detection unit 25 and a determination unit 26 in addition to the configuration of the control device 2-1 shown in FIG. 2. Note that description of the same functions as the constituent elements shown in FIG. 2 will be omitted herein.

The communication unit 22 according to the second embodiment receives a signal for position detection from the capsule 1 via the extracorporeal unit 7.

The position detection unit 25 detects (computes) a position of the capsule 1 based on the signal for position detection that the communication unit 22 has received. In addition, the detected position of the capsule 1 is output to the determination unit 26.

The determination unit 26 determines whether or not the position of the capsule 1 detected by the position detection unit 25 is in the periphery of a pre-specified medicine sparging site, and then outputs the determination result to the control unit 21. Note that the specification of the medicine sparging site may be performed by, for example, displaying a specifying screen on the display unit 23 and then accepting designation of a sparging site from a medical staff in advance. The specification of a medicine sparging site using the specifying screen will be described in detail in “2-2-4. Specification of a medicine sparging site.”

In addition, the control unit 21 according to the second embodiment controls the communication unit 22 such that an actuation signal is transmitted to the rotating magnetic field generator 6 when the determination unit 26 determines that the capsule 1 has reached a periphery of the pre-specified medicine sparging site.

### 2-2-2. Structure of a Capsule-Type Medical Device

Since a basic structure of the capsule 1 included in the medical system according to the second embodiment is the same as the capsule 1 according to the first embodiment, description thereof will be omitted herein. Note that the memory and communication unit 38 of the capsule 1 according to the second embodiment further has a function of transmitting a signal for position detection.

### 2-2-3. Medicine Discharge Process

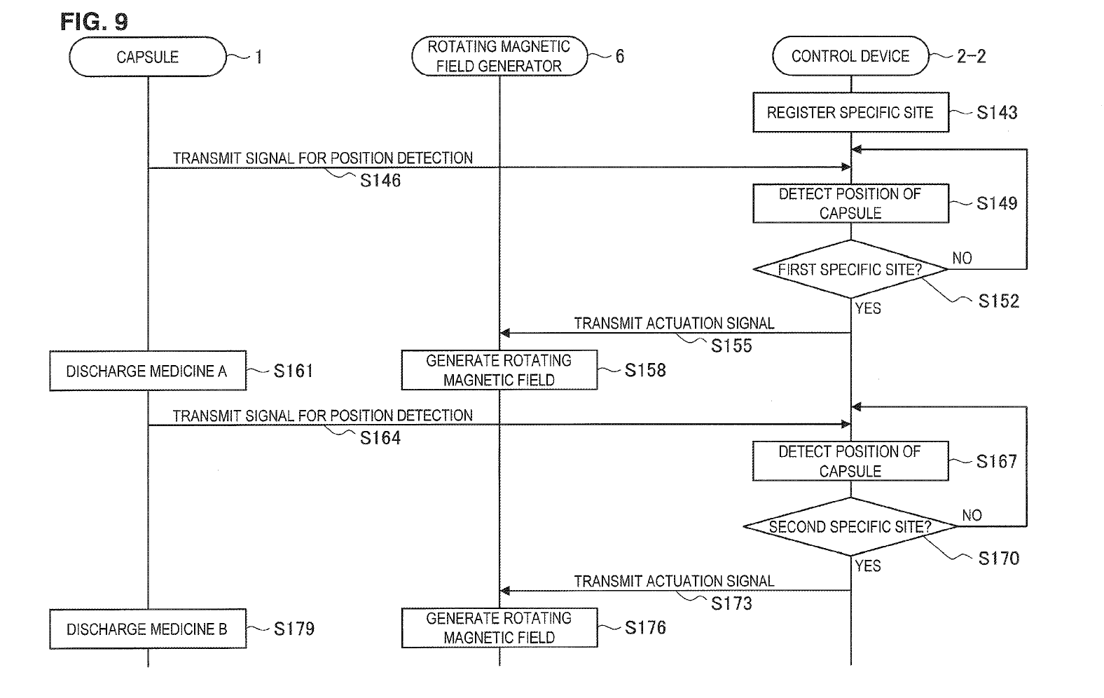

Next, a medicine discharge process according to the present embodiment will be described in detail with reference to FIG. 9. FIG. 9 is a flowchart showing the medicine discharge process according to the second embodiment. As shown in FIG. 9, the control device 2-2 first registers specific sites to which medicines are sparged (discharged) in Step S143. To be more specific, the control device 2-2 memorizes a plurality of specific sites to which medicines are sparged in association with each of the medicines to be sparged to the plurality of specific sites.

Next, in Step S146, a signal for position detection is transmitted from the capsule 1. Next, in Step S149, the position detection unit 25 of the control device 2-2 detects the position of the capsule 1 based on the signal for position detection transmitted from the capsule 1.

Next, in Step S152, the determination unit 26 of the control device 2-2 determines whether or not the position of the capsule 1 detected by the position detection unit 25 is in the periphery of a first specific site registered in Step S143.

Next, when the determination unit 26 determines that the position of the capsule 1 is in the periphery of the first specific site, the control device 2-2 transmits an actuation signal to the rotating magnetic field generator 6 in Step S155.

Next, in Step S158, the rotating magnetic field generator 6 generates a rotating magnetic field according to the received actuation signal as in Step S118 shown in FIG. 7.

Next, in Step S161, the capsule 1 discharges the medicine A as in Step S121 shown in FIG. 7. Accordingly, the medicine A (first medicine) is discharged to the outside of the capsule 1 in the periphery of the pre-registered first specific site, and thereby the medicine A is sparged in the periphery of the first specific site.

Subsequently, in Steps S164 to S179, the same processes as Steps S146 to 161 are performed, and thereby the medicine B (second medicine) is discharged in the periphery of a second specific site.

In other words, after the process of discharging the medicine A shown in Step S161, the capsule 1 continues to transmit the signal for position detection to the control device 2-2 while passing through the inside of the body cavity duct line 29 in Step S164.

Next, in Step S167, the position detection unit 25 of the control device 2-2 detects a position of the capsule 1 based on the signal for position detection transmitted from the capsule 1.

Next, in Step S170, the determination unit 26 of the control device 2-2 determines whether or not the position of the capsule 1 detected by the position detection unit 25 is in the periphery of the second specific site registered in Step S143 described above.

Next, when the determination unit 26 determines that the position of the capsule 1 is in the periphery of the second specific site, the control device 2-2 transmits an actuation signal to the rotating magnetic field generator 6 in Step S173 as in Step S155 described above.

Next, in Step S176, the rotating magnetic field generator 6 generates a rotating magnetic field according to the actuation signal as in Step S158 described above.

Next, in Step S179, the capsule 1 discharges the medicine B as in Step S139 shown in FIG. 7. Accordingly, the medicine B (second medicine) is discharged to the outside of the capsule 1 in the periphery of the pre-registered second specific site, and thereby the medicine B is sparged in the periphery of the second treatment site.

Then, the capsule 1 emitted to the outside of the body is collected or the like by the medical staff, and thereby treatment using the capsule 1 ends.

As described above, in the medicine discharge process according to the second embodiment, by detecting the positions of the capsule 1 and determining whether or not the capsule 1 is in the periphery of specific sites, the first and second medicines can be sequentially discharged automatically from the capsule 1 to desired sites.

### 2-2-4. Specification of a Medicine Sparging Site

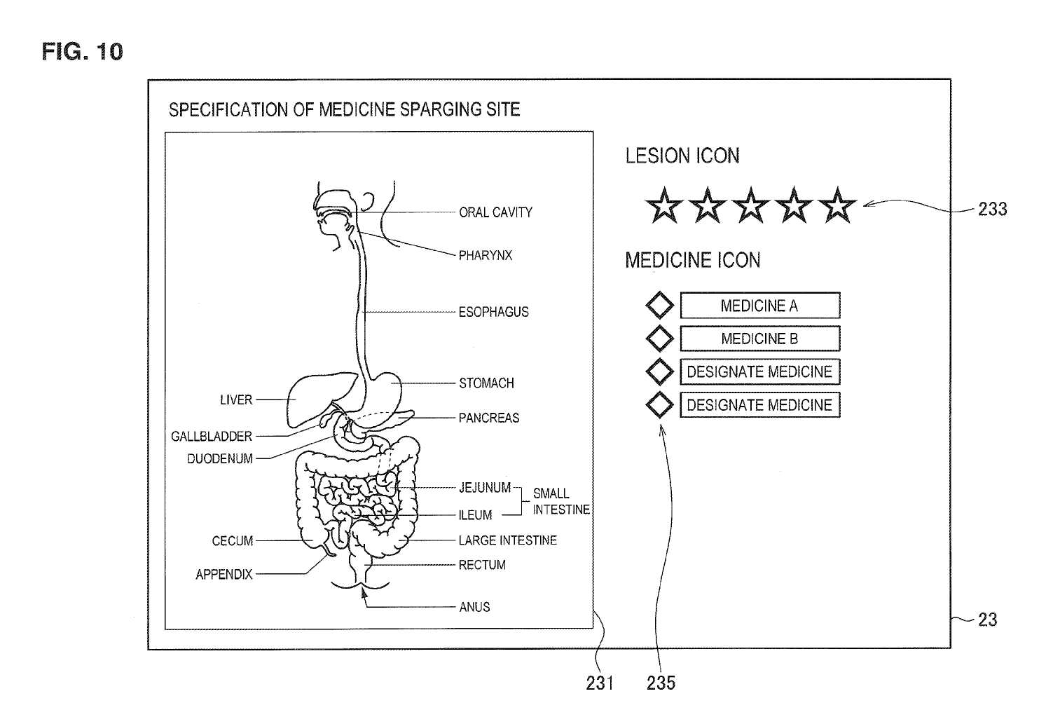

Next, an example of a method for registering a specific site according to the second embodiment will be described in detail with reference to FIGS. 10 to 12. As shown in FIGS. 10 to 12, a medical staff can instantaneously register a specific site to which each medicine is sparged according to a specifying screen displayed on the display unit 23 of the control device 2-2.

FIG. 10 is a diagram showing an example of a medicine sparging site specifying screen displayed on the display unit 23 of the control device 2-2 according to the second embodiment. As shown in FIG. 10, the medicine sparging site specifying screen includes a site image 231 that shows each intracorporeal site, lesion icons 233, and medicine icons 235.

The site image 231 may be an image in which the illustration of each site is associated with its name as shown in FIG. 10. Note that, in the example shown in FIG. 10, the stereotypical illustration of sites of a body is displayed as the site image 231, however, an actual position of the body of the subject 3 is substantially fixed, and thus the control unit 21 of the control device 2-2 has already recognized the absolute positions (coordinates of the positions) of organs (sites). Therefore, the control unit 21 can compute the coordinates of the positions of the sites of the subject 3 that correspond to each of the sites shown in the site image 231.

Each of the lesion icons 233 is an icon for specifying a site to which a medicine is to be sparged.

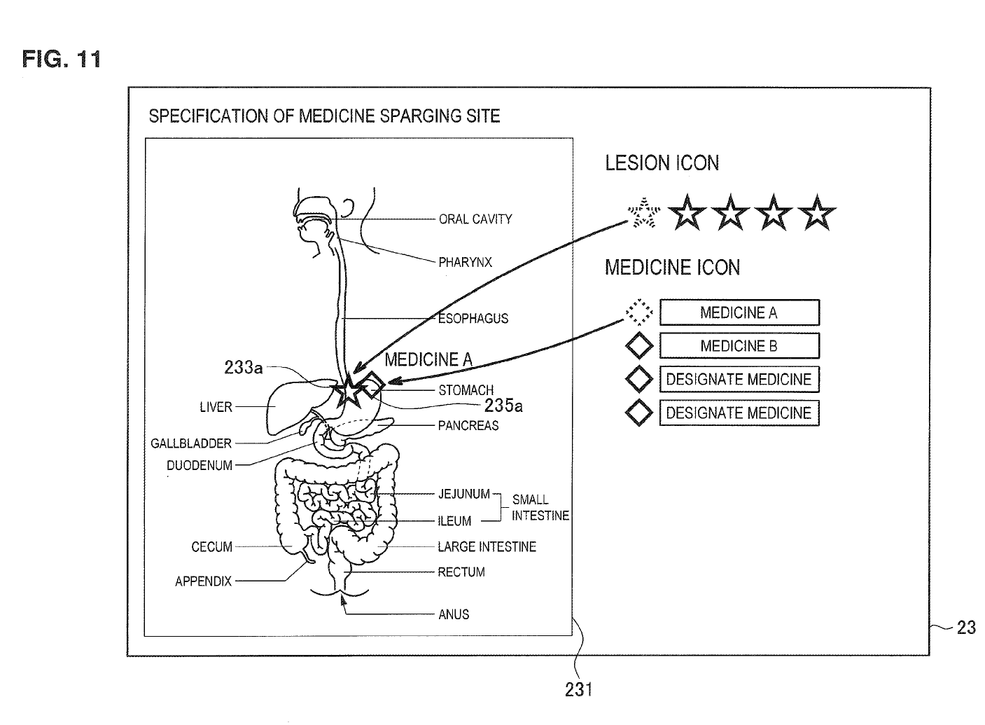

Each of the medicine icons 235 is an icon for designating a medicine to be sparged. Here, the capsule 1 according to the present embodiment has the plurality of storage units 40 and 41 in which different medicines can be stored as shown in FIGS. 4 to 6. Thus, as shown in FIG. 11, each of the medicine icons 235 is displayed in association with a medicine.

Subsequently, registration of the first specific site and designation of a medicine to be sparged to the first specific site will be described with reference to FIG. 11. As shown in FIG. 11, a medical staff selects a lesion icon 233a, and moves the icon to a periphery of a desired site through drag-and-drop manipulation. Further, the medical staff selects a medicine icon 235a of a medicine to be sparged in the site to which the lesion icon 233a has been moved and then moves the medicine icon in the periphery of the lesion icon 233a.

Accordingly, the control unit 21 of the control device 2-2 computes the coordinates of an actual position in the body of the subject 3 that corresponds to the first specific site based on the selection and movement manipulation of the lesion icon 233a performed by the medical staff. In addition, the control unit 21 registers the coordinates of the position of the first specific site in association with the designated medicine based on the selection and movement manipulation of the medicine icon 235a performed by the medical staff.

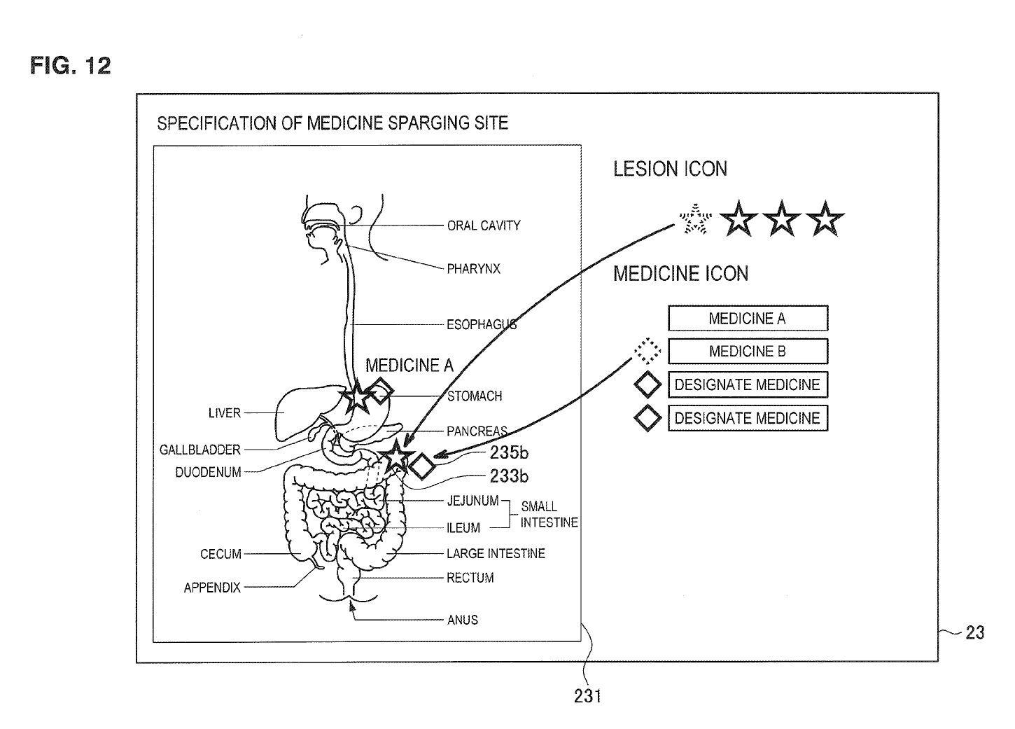

Subsequently, registration of the second specific site and designation of a medicine to be sparged in the second specific site are also performed in the same manner. To be specific, the medical staff selects a lesion icon 233b and moves the icon to a periphery of a desired site through drag-and-drop manipulation as shown in, for example, FIG. 12. Further, the medical staff selects a medicine icon 235b of a medicine to be sparged in the site to which the lesion icon 233b has been moved and then moves the medicine icon in the periphery of the lesion icon 233b.

Accordingly, the control unit 21 of the control device 2-2 computes the coordinates of an actual position in the body of the subject 3 that corresponds to the second specific site based on the selection and movement manipulation of the lesion icon 233b performed by the medical staff. In addition, the control unit 21 registers the coordinates of the position of the second specific site in association with the designated medicine based on the selection and movement manipulation of the medicine icon 235b performed by the medical staff.

Hereinabove, as the second embodiment of the present disclosure, the medical system in which a position of the capsule 1 is detected, whether or not the capsule 1 reaches the periphery of a pre-specified site is determined, and a medicine is sparged from the capsule 1 according to the determination result has been described.

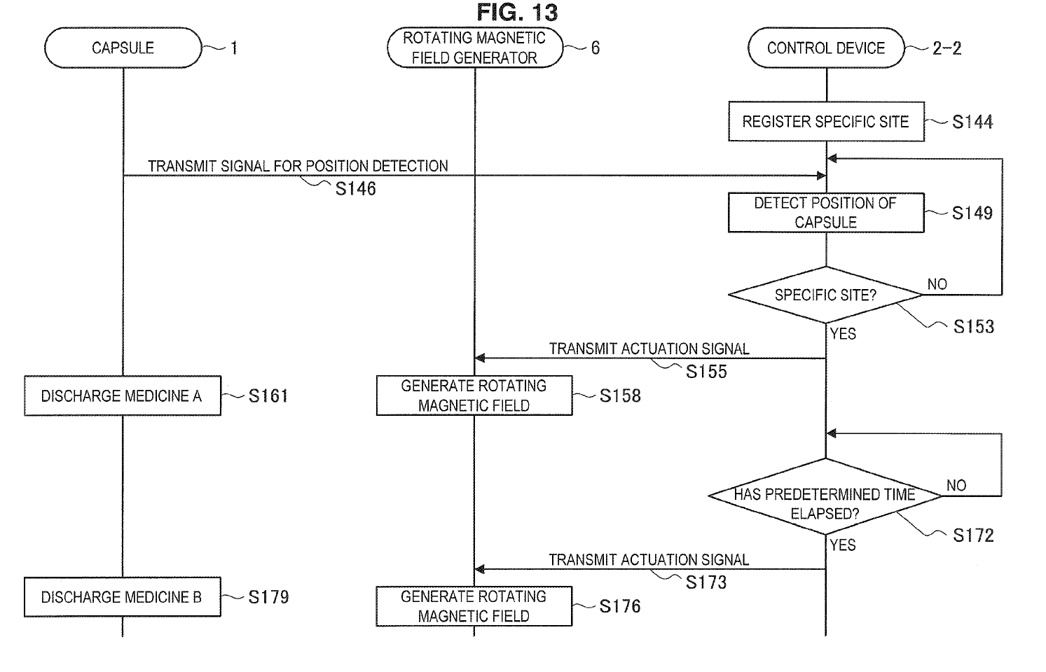

Note that, in the second embodiment, when the capsule 1 is determined to have reached a periphery of the second specific site after the medicine A (first medicine) has been discharged in the periphery of the first specific site, the medicine B (second medicine) is discharged. The discharge timing of the second medicine according to the present disclosure, however, is not limited thereto. Hereinbelow, another example with regard to a discharge timing of the second medicine will be described with reference to FIG. 13.

### Modified Example 1

FIG. 13 is a flowchart showing a medicine discharge process according to a modified example of the second embodiment. As shown in FIG. 13, the control device 2-2 first registers a specific site to which a medicine is sparged in Step S144. To be more specific, the control device 2-2 memorizes one specific site to which a medicine is sparged based on, for example, an input of manipulation by a medical staff.

Subsequently, since S146 to S149 are the same processes as the steps of the same numbers shown in FIG. 9 described above, description thereof will be omitted herein.

Next, in Step S153, the determination unit 26 of the control device 2-2 determines whether or not a position of the capsule 1 detected by the position detection unit 25 is in the periphery of the specific site registered in Step S144 described above.

Subsequently, in S155 to S161, the same processes as the steps with the same numbers shown in FIG. 9 described above are executed. In other words, the medicine A (first medicine) is discharged to the outside of the capsule 1 in the periphery of the pre-registered specific site, and thereby the medicine A is discharged to the periphery of the specific site.

Next, in Step S172, the control device 2-2 determines whether or not a predetermined time has elapsed after the medicine A was discharged from the capsule 1 (herein, it may be after an actuation signal is transmitted to the rotating magnetic field generator 6).

Then, when the predetermined time is determined to have elapsed, in S173 to S179, the same processes as the steps with the same numbers shown in FIG. 9 described above are subsequently executed. In other words, the medicine B (second medicine) is discharged to the outside of the capsule 1, and thereby the medicine B is sparged.

As described above, in the medical system according to modified example 1, the second medicine can be discharged after the predetermined time elapses from the discharge of the first medicine to the specific site. As described above, by sparging a plurality of medicines to one site or different sites with time differences, an effect that is difficult to obtain when a subject ingests a plurality of medicines at the same time for therapy can be exhibited.

### 2-3. Third Embodiment

The capsule 1 of the first and second embodiments described above has the structure described with reference to FIGS. 4 to 6, and discharges the medicine A first and then the medicine B based on the structure. A structure of a capsule-type medical device of the present disclosure, however, is not limited to the example shown in FIG. 4 or the like, and may be a structure in which, for example, any of the medicines A and B can be discharged first (or only one thereof can be discharged) according to a situation.

Thus, in a third embodiment of the present disclosure, a medical system using a capsule-type medical device 70 that has a structure in which a medicine among the medicine A and medicine B is discharged according to a discharge signal (control signal) from outside will be described. The medical system according to the present embodiment includes the capsule-type medical device 70 ingested by the subject 3 and the control device 2-2 of the second embodiment described above. Hereinbelow, after a structure of the capsule-type medical device 70 of the present embodiment is described, a medicine discharge process of the present embodiment will be described in detail.

### 2-3-1. Structure of a Capsule-Type Medical Device

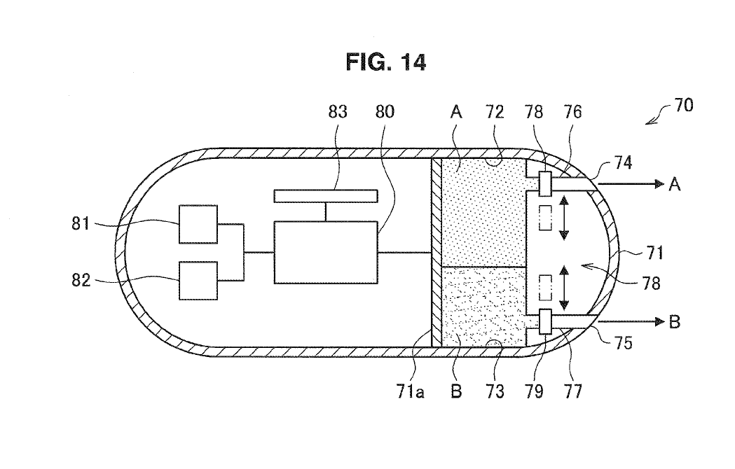

FIG. 14 is a diagram showing an internal structure of main parts of the capsule-type medical device 70 according to the third embodiment. As shown in FIG. 14, the capsule-type medical device 70 of the present embodiment (hereinafter referred to as a capsule 70) has a battery 83, a control unit 80, a reception unit 81, and a transmission unit 82 on the left side of a wall portion 71a inside a capsule-shaped housing 71. Note that the housing 71 is formed of plastic or the like so as to hermetically seal the inside.

In addition, on the right side of the wall portion 71a, storage units 72 and 73 in which medicines are stored, medicine discharge ports 74 and 75 formed on the outer surface of the housing 71, medicine discharge duct lines 76 and 77 each causing a storage unit to communicate with a medicine discharge port, and opening-closing valves 78 and 79 that respectively open and close the duct lines 76 and 77 are provided. Note that a plurality of medicine discharge ports 74 and 75 may be formed around an axis on one end side of the housing 71.

Hereinbelow, each constituent element of the capsule 70 will be described. The battery 83 is, for example, a button type battery which supplies power to each of the constituent elements of the control unit 80, the reception unit 81, and the transmission unit 82.

The control unit 80 has a function of controlling the entire capsule 70. In addition, the control unit 80 (medicine discharge unit) of the present embodiment causes the opening-closing valve 78 or 79 to actuate according to a discharge signal (control signal) received by the reception unit 81 to discharge the first or second medicine.

The reception unit 81 has a function of receiving data from an external device. For example, the reception unit 81 receives the discharge signal from the control device 2-2 (via the extracorporeal unit 7). In addition, the reception unit 81 outputs the received discharge signal to the control unit 80. The transmission unit 82 has a function of transmitting data to an external device. For example, the transmission unit 82 transmits a radio wave as position information indicating a position of the capsule (signal for position detection) to the control device 2-2 (via the extracorporeal unit 7).

Note that, in the example shown in FIG. 14, the reception unit 81 and the transmission unit 82 are shown in separate blocks, but the configuration of the capsule 70 is not limited to the example shown in FIG. 14, and, for example, a communication unit having a reception function and a transmission function may be provided.

The storage unit 72 stores the first medicine (herein, the medicine A). In addition, the medicine discharge duct line 76 is connected to the storage unit 72 as shown in FIG. 14. In addition, the opening-closing valve 78 that can move to open and close the medicine discharge duct line 76 is provided as described above.

On the other hand, the storage unit 73 stores the second medicine (herein, the medicine B). In addition, the medicine discharge duct line 77 is connected to the storage unit 73 as shown in FIG. 14. In addition, the opening-closing valve 79 that can move to open and close the medicine discharge duct line 77 is provided as described above.

In the capsule 70 having such a structure, both of the opening-closing valves 78 and 79 are respectively set in positions for blocking both the duct lines 76 and 77 in an initial state. Then, when the control unit 80 controls such that the opening-closing valves 78 and 79 respectively open both duct lines 76 and 77 according to the discharge signal received by the reception unit 81 from the control device 2-2, the medicines are discharged.

For example, when the reception unit 81 receives a first discharge signal, the control unit 80 controls the opening-closing valve 78 in order to open the medicine discharge duct line 76, and is thereby able to discharge the medicine A. In addition, when the reception unit 81 receives a second discharge signal, the control unit 80 controls the opening-closing valve 79 in order to open the medicine discharge duct line 77, and is thereby able to discharge the medicine B.

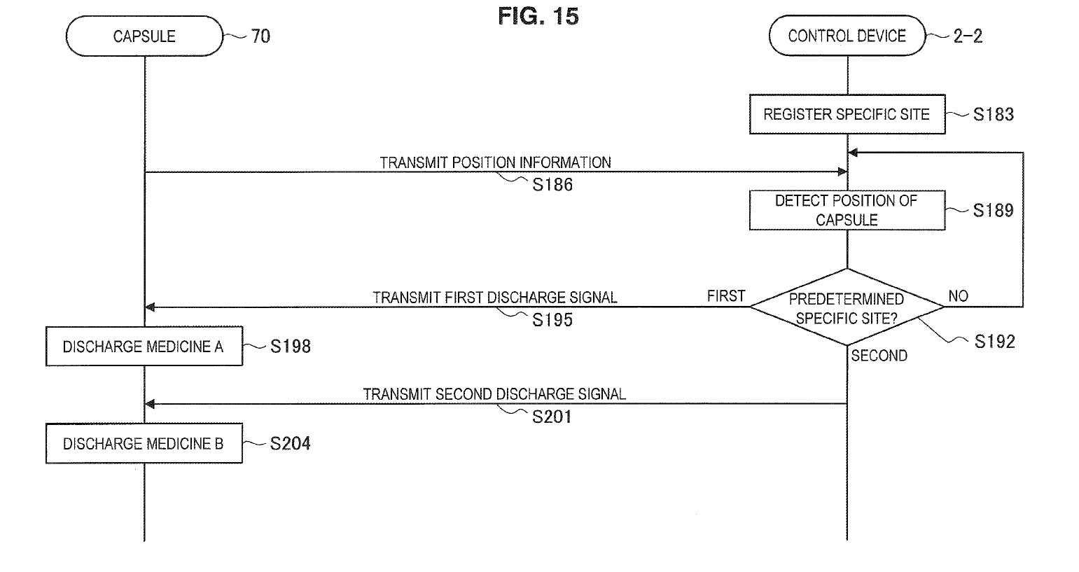

Hereinabove, the structure of the capsule 70 according to the third embodiment has been described in detail. Next, a medicine discharge process according to the present embodiment will be described with reference to FIG. 15.

### 2-3-2. Medicine Discharge Process

FIG. 15 is a flowchart showing the medicine discharge process according to the third embodiment. As shown in FIG. 15, the control device 2-2 first registers specific sites to which the medicines are sparged in Step S183. To be more specific, the control device 2-2 memorizes the plurality of specific sites to which the medicines are sparged in association with each of the medicines to be sparged to the plurality of specific sites based on, for example, an input of manipulation performed by a medical staff.

Next, in Step S186, the capsule 70 taken by the subject 3 transmits position information to the control device 2-2.

Next, in Step S189, the position detection unit 25 of the control device 2-2 detects a position of the capsule 70 based on the position information.

Next, in Step S192, the determination unit 26 of the control device 2-2 determines whether or not the position of the capsule 70 detected by the position detection unit 25 is in the periphery of the pre-registered specific sites.

Next, when the capsule 70 is determined to have reached the periphery of the first specific site in Step S192, the control device 2-2 transmits the first discharge signal to the capsule 70 in Step S195. Next, in Step S198, the capsule 70 receives the first discharge signal and then controls such that the opening-closing valve 78 allows the first medicine (medicine A) to be discharged.

On the other hand, when the capsule 70 is determined to have reached the periphery of the second specific site in Step S192, the control device 2-2 transmits the second discharge signal to the capsule 70 in Step S204. Next, in Step S204, the capsule 70 receives the second discharge signal and then controls such that the opening-closing valve 79 allows the second medicine (medicine B) to be discharged.

Note that the capsule 70 continuously performs transmission of the position information shown in Step S186 described above while passing through the inside of the body of the subject 3. In addition, the processes of Steps S189 and S192 by the control device 2-2 described above are repeatedly performed based on the position information transmitted from the capsule 70. Thus, when the capsule 70 discharges the medicine A in Step S198 described above and then the control device 2-2 transmits the second discharge signal in Step S201, for example, the capsule 70 discharges the medicine B in Step S204.

As described above, according to the third embodiment, a medicine among a plurality of medicines can be selectively discharged according to a discharge signal received from outside. Here, the first and second discharge signals (control signals) transmitted by the control device 2-2 may include medicine identification information. In this case, if the identification information of a received discharge signal is legitimate (for example, if identification information of the medicine stored in the capsule coincides with identification information included in a discharge signal), the capsule 70 controls such that the medicine is discharged. Accordingly, even when the subject 3 mistakenly ingests a capsule in which a wrong medicine is stored, the wrong medicine can be prevented from being sparged.

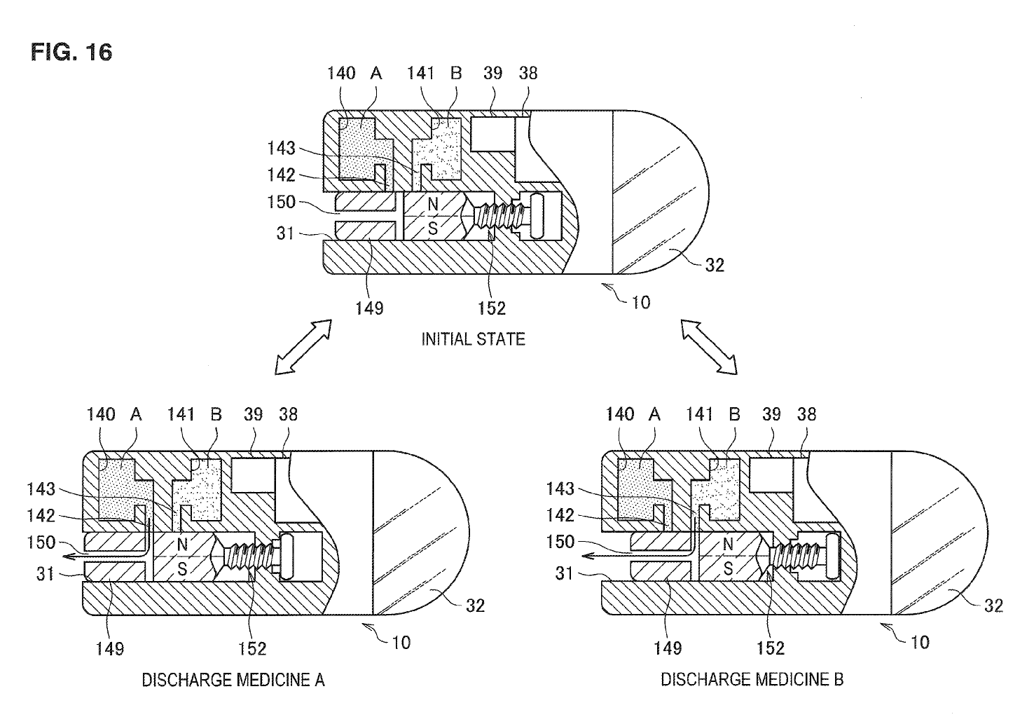

Note that the structure of the capsule-type medical device according to the third embodiment is not limited to the structure shown in FIG. 14. For example, the structure in which any of the medicines A and B is discharged first (or only one thereof is discharged) according to a situation may be the structure shown in FIG. 16. A capsule-type medical device 10 (hereinafter referred to also as a capsule 10) shown in FIG. 16 is a modified example of the capsule 1 of the first and second embodiments. Hereinbelow, a structure of the capsule 10 according to modified example 2 will be described with reference to FIG. 16.

### Modified Example 2

FIG. 16 is an example showing a main internal structure of the capsule-type medical device 10 according to modified example 2. As shown in FIG. 16, the capsule 10 has a storage unit 140 in which the medicine A is stored and a storage unit 141 in which the medicine B is stored.

In addition, the capsule 10 has a moving body 152 that includes a cylinder portion 149 in which a T-shaped hole 150 is formed. The moving body 152 is rotated and moves in the longitudinal direction of the capsule 10 according to rotating magnetic fields generated by the electromagnets 14 of the rotating magnetic field generator 6 shown in FIG. 3 to open and close an opening 142 of the storage unit 140 and an opening 143 of the storage unit 141 using the cylinder portion 149.

As shown in FIG. 16, in an initial state, the capsule 10 blocks the opening 142 of the storage unit 140 and the opening 143 of the storage unit 141 using the cylinder portion 149.

Next, when the moving body 152 is rotated and moves on the storage unit 140 side according to the rotating magnetic field, the T-shaped hole 150 provided in the cylinder portion 149 communicates with the opening 142 as shown in FIG. 16, and thereby the medicine A is discharged to the outside of the capsule 10.

On the other hand, when the moving body 152 is rotated and moves on the storage unit 141 side according to the rotating magnetic field, the T-shaped hole 150 provided in the cylinder portion 149 communicates with the opening 143 as shown in FIG. 16, and thereby the medicine B is discharged to the outside of the capsule 10.

Here, the rotation and movement direction of the moving body 152 can be changed by changing a rotation direction of a rotating magnetic field generated by the rotating magnetic field generator 6. In addition, the control device 2-2 may detect an orientation (attitude) of the capsule 10 and then transmit an actuation signal that includes an instruction of an orientation of a rotating magnetic field to the rotating magnetic field generator 6 according to the detected orientation of the capsule and a medicine (medicine A or B) to be discharged. Note that the control device 2-2 may detect the orientation of the capsule 10 based on orientation information output from a direction sensor (not illustrated) that the capsule 10 has, or may detect the orientation by actually moving the moving body 152 slightly by applying a magnetic field for a predetermined time.

### 2-4. Fourth Embodiment

In the first to third embodiments described above, a medicine to be discharged to each specific site is decided in advance by a medical staff, but there are also cases in which it is difficult to decide an efficacious medicine before a condition of a specific site (lesion) is ascertained using an endoscope or the like. With respect to a prescribed medicine for a stomachache, there are cases in which a medicine that facilitates secretion of gastric acid is appropriate, and cases in which a medicine that inhibits secretion of gastric acid is appropriate depending on a situation.

Thus, a fourth embodiment of the present disclosure proposes a medical system that includes a capsule-type medical device that selects a medicine to be discharged among a plurality of medicines according to a condition of a specific site (lesion). Note that the medical system of the present embodiment includes the capsule-type medical device and the control device 2-2 of the second embodiment described above.

In addition, as a medicine selection method according to a condition of a specific site according to the present embodiment, for example, a method in which a condition of a specific site is diagnosed and a medicine to be discharged is selected according to the diagnosis result and a method in which a condition of a specific site is detected and a medicine to be discharged is selected according to the detection result are exemplified. Hereinbelow, the capsule-type medical device that diagnoses a condition and a capsule-type medical device that detects a condition will be described in order.

### 2-4-1. Diagnosis of a Condition

Configuration

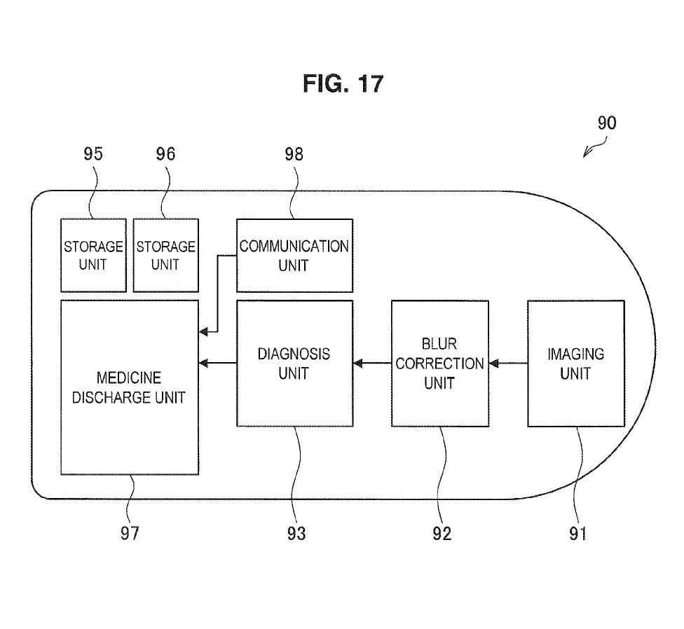

FIG. 17 is a block diagram showing a configuration of a capsule-type medical device 90 that performs diagnosis of a condition according to the fourth embodiment. As shown in FIG. 17, the capsule-type medical device 90 (hereinafter referred to as a capsule 90) has an imaging unit 91, a blur correction unit 92, a diagnosis unit 93, a medicine discharge unit 97, a plurality of storage units 95 and 96, and a communication unit 98.

The imaging unit 91 images a periphery of a specific site or the like, and then outputs a captured image to the blur correction unit 92. The blur correction unit 92 corrects the received captured image through image processing. Note that the blur correction according to the present embodiment is not limited to correction through image processing, and may be lens shift correction, CCD shift correction, or correction through alteration of a liquid lens.

In addition, the imaging unit 91 of the present embodiment may output the captured image to the diagnosis unit 93 directly without performing blur correction.

The diagnosis unit 93 diagnoses a condition of the periphery of the specific site based on the captured image, and then outputs the diagnosis result to the medicine discharge unit 97. Here, the diagnosis unit 93 of the present embodiment performs diagnosis based on the captured image of the inside of a body, but the function of the diagnosis unit 93 of the present embodiment is not limited thereto. When, for example, the capsule 90 further has a sampling unit that samples a biological tissue from a periphery of an intracorporeal site, the diagnosis unit 93 may diagnose a condition based on the biological tissue sampled by the sampling unit. In addition, when the capsule 90 further has a sensor that detects a condition of the inside of a body, the diagnosis unit 93 may diagnose a condition based on a predetermined value (for example, a pH value) detected by the sensor in the periphery of an intracorporeal site.

The communication unit 98 has a function of performing transmission and reception of data with an external device. For example, the communication unit 98 transmits position information to the control device 2-2 and also receives a discharge signal (control signal) from the control device 2-2.

The storage unit 95 stores, for example, the first medicine (medicine A). The medicine A stored in the storage unit 95 passes a duct line that is not illustrated and is discharged to the outside of the capsule 90. In addition, the storage unit 96 stores, for example, the second medicine (medicine B). The medicine B stored in the storage unit 96 passes through a duct line that is not illustrated and is discharged to the outside of the capsule 90.

Each of the duct lines through which the medicines are discharged to the outside of the capsule 90 from the storage units 95 and 96 is opened and closed by the medicine discharge unit 97.

When a communication unit 98 receives a discharge signal, the medicine discharge unit 97 controls such that the duct line of the storage unit 95 or the storage unit 96 is opened so that the medicine in the storage unit 95 or the storage unit 96 is discharged based on a diagnosis result of a condition in the periphery of a specific site output from the diagnosis unit 93. Note that the detailed structure in which the medicine A or the medicine B is discharged according to a diagnosis result may be the structure of the capsule 70 described with reference to FIG. 14 in the third embodiment above.

Operation Process

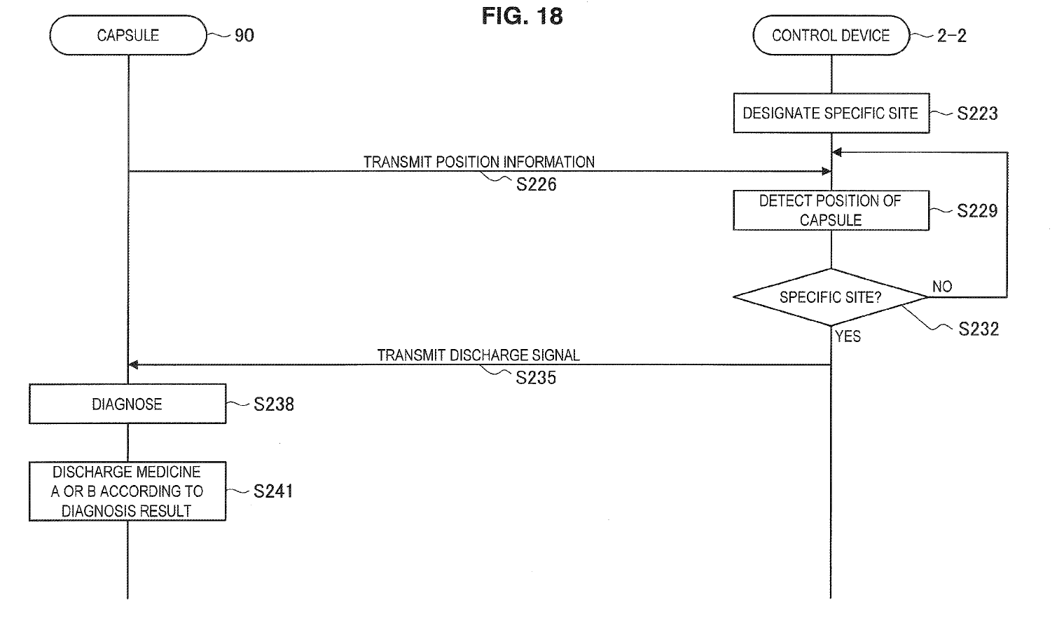

Next, a medicine discharge process according to the present embodiment will be described with reference to FIG. 18. FIG. 18 is a flowchart showing the medicine discharge process in accordance with diagnosis of a condition according to the fourth embodiment.

As shown in FIG. 18, first, the control device 2-2 and the control device 2-2 register specific sites to which medicines are to be discharged in Step S223. To be more specific, the control device 2-2 memorizes one specific site to which a medicine is to be discharged based on, for example, an input of manipulation performed by a medical staff.

Next, since S226 to S229 are the same processes as Steps S186 to S189 shown in FIG. 15 described above, description thereof is omitted herein.

Next, in Step S232, the determination unit 26 of the control device 2-2 determines whether or not a position of the capsule 90 detected by the position detection unit 25 is in the periphery of the specific site registered in Step S223.

Next, when the capsule 90 is determined to have reached the periphery of the specific site in Step S232, the control device 2-2 transmits a discharge signal to the capsule 90 in Step S235.

Next, in Step S238, the capsule 90 diagnoses a condition of the periphery of the specific site using the diagnosis unit 93.

Next, in Step S241, the capsule 90 controls such that the medicine A or the medicine B is discharged according to the diagnosis result obtained by the diagnosis unit 93.

Hereinabove, selection of a medicine according to diagnosis of a condition according to the fourth embodiment has been described. Next, selection of a medicine according to detection of a condition in another example of the present embodiment will be described in detail.

### 2-4-2. Detection of a Condition

Configuration

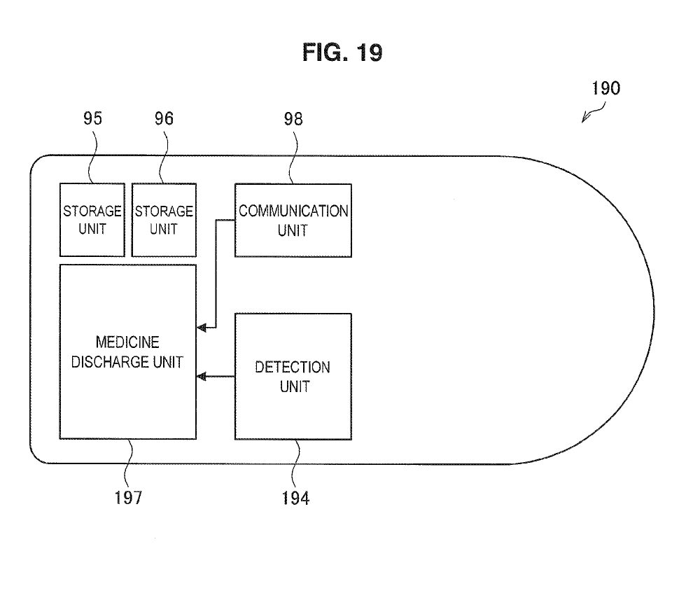

FIG. 19 is a block diagram showing a configuration of a capsule-type medical device 190 that performs detection of a condition according to the fourth embodiment. As shown in FIG. 19, the capsule-type medical device 190 (hereinafter referred to as a capsule 190) has a detection unit 194, a medicine discharge unit 197, the plurality of storage units 95 and 96, and the communication unit 98. Note that, since the plurality of storage units 95 and 96 and the communication unit 98 have been described above with reference to FIG. 17, description thereof is omitted herein.

The detection unit 194 is a sensor that detects a condition of a periphery of a specific site, and outputs the detection result to the medicine discharge unit 197. For example, the detection unit 194 may be a sensor that detects a pH value of gastric acid.

When the communication unit 98 receives a discharge signal, the medicine discharge unit 197 controls such that the duct line of the storage unit 95 or the storage unit 96 is opened so that the medicine in the storage unit 95 or the storage unit 96 is discharged based on the detection result of the condition of the periphery of the specific site output from the detection unit 194. Note that the detailed structure in which the medicine A or the medicine B is discharged according to a diagnosis result may be the structure of the capsule 70 described with reference to FIG. 14 in the third embodiment above.

Operation Process

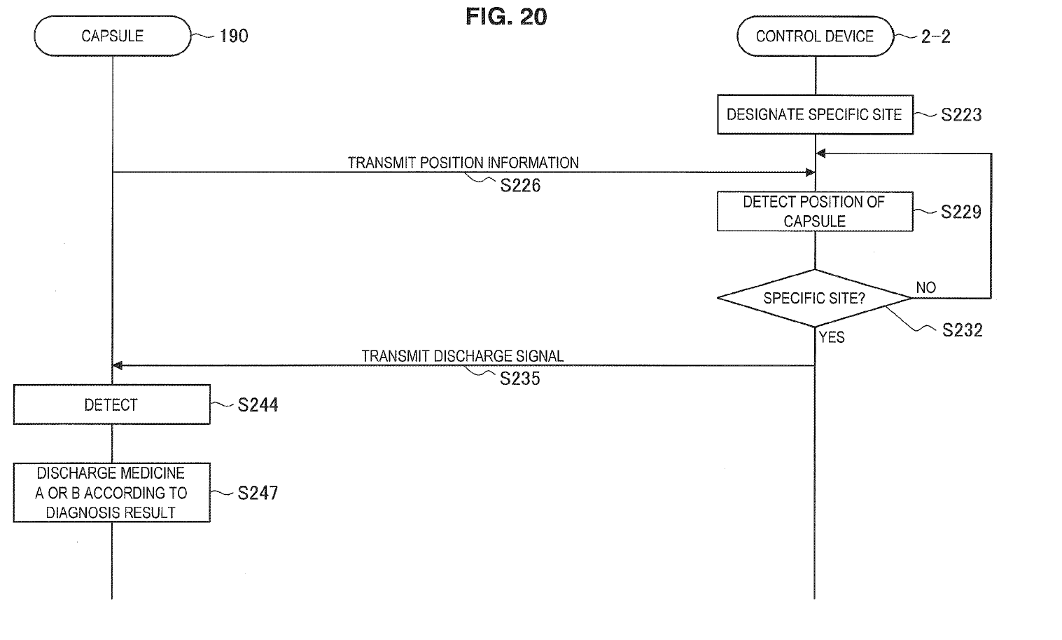

Next, a medicine discharge process according to the present embodiment will be described with reference to FIG. 20. FIG. 20 is a flowchart showing the medicine discharge process in accordance with the detection of a condition according to the fourth embodiment.

Since S226 to S235 shown in FIG. 20 are the same processes as the steps with the same numbers shown in FIG. 18, description thereof is omitted herein.

Next, in Step S244, the capsule 190 detects a condition of the periphery of the specific site using the detection unit 194.

Next, in Step S247, the capsule 190 controls such that the medicine A or the medicine B is discharged according to the detection result obtained by the detection unit 194.

As described above, according to the medical system of the fourth embodiment, a medicine to be discharged among a plurality of medicines can be selected in accordance with a condition of a specific site (lesion). Accordingly, when the capsule reaches a lesion, an appropriate medicine can be discharged in accordance with a condition of the lesion even when it is difficult to decide a medicine to be discharged in advance.

### 3. Conclusion

As described above, in the medical system of the present embodiments, the capsule-type medical device that has a plurality of storage units in which medicines are stored can selectively discharge at least one among the plurality of medicines in the inside of the body of the subject 3.

More specifically, according to the first embodiment, for example, a desired medicine among a plurality of medicines can be discharged to a site that a medical staff desires.

In addition, according to the second embodiment, whether or not the capsule has reached a periphery of a pre-specified site is automatically determined, and a medicine designated in advance for the specific site can be selectively discharged.

In addition, according to the third embodiment, any of the medicines A and B can be discharged first (or only one thereof can be discharged) according to a situation.

In addition, according to the fourth embodiment, a medicine among a plurality of medicines can be selectively discharged according to (appropriate for) a condition of a specific site.

Hereinabove, the preferred embodiments of the present disclosure have been described above in detail with reference to the accompanying drawings, whilst the present technology is not limited to the above examples. A person skilled in the related art of the present disclosure may find various alternations and modifications within the scope of the technical gist of the appended claims, and it should be understood that they will naturally come under the technical scope of the present disclosure.

In the fourth embodiment described above, for example, the diagnosis unit 93 that the capsule 90 has diagnoses a condition of a specific site, but the medical system according to the present disclosure is not limited thereto, and may be a medical system in which a medicine is selectively discharged based on, for example, a diagnosis result made by a medical staff.

To be more specific, a captured image of a periphery of a specific site captured by the capsule 90 is first transmitted to the control device 2-2 and then displayed on the display unit 23. Accordingly, after a medical staff diagnoses a condition of the periphery of the specific site, an appropriate medicine can be selected. The control device 2-2 transmits a discharge signal (control signal) of the selected medicine to the capsule 90 in response to an input of manipulation performed by the medical staff, and then the capsule 90 selectively discharges the medicine in accordance with the received discharge signal.

In addition, in each of the embodiments described above, the capsule 1 and the control device 2-1 or 2-2 perform transmission and reception of data via the extracorporeal unit 7, but the medical system of the embodiment is not limited thereto. For example, the capsule 1 and the control device 2-1 or 2-2 may perform transmission and reception of data directly.

In addition, each of the embodiments has been described using the capsule-type medical device, but a medical device of the present disclosure is not limited to a capsule type, and for example, may be a medical device integrated with an endoscope which selectively discharges a plurality of medicines from a tip end part of the endoscope.

In addition, a captured image of an intracorporeal site captured by the imaging unit that the capsule has in each of the embodiments may be used for measuring efficacy after administration.

In addition, the capsule of each of the embodiments described above may have a sampling unit that samples a biological tissue from a site of the inside of a body. The sampling unit samples a biological tissue from a periphery of a specific site after, for example, discharge of a medicine, and the sampled biological tissue is used for measuring efficacy after administration after collection of the capsule.

Furthermore, in each of the embodiments described above, each of a plurality of medicines is discharged, but the medical device of the present disclosure can mix the medicines to be administered by controlling a discharge ratio of the plurality of medicines. For example, as a modified example of the medical device of the fourth embodiment described above that discharges a medicine in accordance with a condition of a specific site, a medical device that can control a discharge ratio of a plurality of medicines in accordance with the condition of the specific site is exemplified. By discharging each of the plurality of medicines at a ratio in accordance with the condition of the specific site substantially at the same time or with a predetermined time difference, the medical device can administer a mixed medicine according to the condition.

Additionally, the present technology may also be configured as below.

(1)

A medical device including:

a plurality of storage units each configured to store a different medicine; and

a medicine discharge unit configured to selectively discharge at least one of a first medicine and a second medicine, each of which is stored in the plurality of storage units, when the medical device reaches a periphery of a predetermined site of an intracorporeal site of a subject.

(2)

The medical device according to (1), wherein the medicine discharge unit discharges the first medicine when the medical device reaches the periphery of the predetermined site and then discharges the second medicine a predetermined time after the discharge of the first medicine.

(3)

The medical device according to (1) or (2), wherein the medicine discharge unit discharges the first medicine when the medical device reaches a periphery of a first site and discharges the second medicine when the medical device reaches a periphery of a second site.

(4)

The medical device according to any one of (1) to (3), further including:

a detection unit configured to detect a condition of the periphery of the predetermined site when the medical device reaches the periphery of the predetermined site,

wherein the medicine discharge unit selectively discharges at least one of the first medicine and the second medicine in accordance with a detection result obtained by the detection unit.

(5)

The medical device according to any one of (1) to (4), further including:
a diagnosis unit configured to diagnose a condition of the periphery of the predetermined site when the medical device reaches the periphery of the predetermined site,

wherein the medicine discharge unit selectively discharges at least one of the first medicine and the second medicine in accordance with a diagnosis result obtained by the diagnosis unit.

(6)

The medical device according to any one of (1) to (5), further including:

an imaging unit configured to image the intracorporeal site of the subject.

(7)

The medical device according to any one of (1) to (6), including:
a sampling unit configured to sample a biological tissue from the intracorporeal site of the subject.
   (8)

A medical system including:

a medical device including
a plurality of storage units each configured to store a different medicine, and
a medicine discharge unit configured to selectively discharge at least one of a first medicine and a second medicine, each of which is stored in the plurality of storage units; and

a control device including
a position detection unit configured to detect a position of the medical device, and
a control unit configured to perform control in a manner that a control signal for causing at least one of the first medicine and the second medicine to be discharged is transmitted to the medical device when the medical device is determined to have reached a periphery of a predetermined site of an intracorporeal site of a subject based on the position detected by the position detection unit.
   (9)

The medical system according to (8), wherein, when medicine identification information included in the control signal transmitted from the control device is legitimate, the medicine discharge unit discharges at least one of the first medicine and the second medicine according to the control signal.

(10)

A program causing a computer to execute:
a process of selectively discharging at least one of a first medicine and a second medicine, each of which is stored in a plurality of storage units each storing a different medicine when a medical device reaches a periphery of a predetermined site of an intracorporeal site of a subject.

### REFERENCE SIGNS LIST

1, 10, 70, 90, 190 capsule-type medical device
2-1, 2-2 control device
3 subject
6 rotating magnetic field generator
7 extracorporeal unit
12 drive circuit
14 electromagnet
21 control unit
22 communication unit
23 display unit
24 manipulation input unit
25 position detection unit
26 determination unit
29 body cavity duct line
30 external case
31 opening
32 transparent cover
34 imaging optical system
36 image sensor
37 control unit
38 communication unit
39 battery
40, 41, 140, 141 storage unit
42, 43, 142, 143 opening
44 first recess portion
45 screw hole (female screw)
46 second recess portion
47 moving body storage unit
48 screw portion (male screw portion)
49, 149 cylinder portion
50, 150 T-shaped hole
51 stopper
52, 152 moving body
54 medicine discharge unit
71 housing
71a wall portion
72, 73 storage unit
74, 75 medicine discharge port
76 medicine discharge duct line
76, 77 medicine discharge duct line
78, 79 opening-closing valve
80 control unit (medicine discharge unit)
81 reception unit
82 transmission unit
83 battery
91 imaging unit
92 blur correction unit
93 diagnosis unit
95, 96 storage unit
97, 197 medicine discharge unit
98 communication unit
194 detection unit
231 site image
233, 233a, 233b lesion icon
235, 235a, 235b medicine icon

1. A medical device comprising:
a plurality of storage units each configured to store a different medicine; and
a medicine discharge unit configured to selectively discharge at least one of a first medicine and a second medicine, each of which is stored in the plurality of storage units, when the medical device reaches a periphery of a predetermined site of an intracorporeal site of a subject.

2. The medical device according to claim 1, wherein the medicine discharge unit discharges the first medicine when the medical device reaches the periphery of the predetermined site and then discharges the second medicine a predetermined time after the discharge of the first medicine.

3. The medical device according to claim 1, wherein the medicine discharge unit discharges the first medicine when the medical device reaches a periphery of a first site and discharges the second medicine when the medical device reaches a periphery of a second site.

4. The medical device according to claim 1, further comprising:
a detection unit configured to detect a condition of the periphery of the predetermined site when the medical device reaches the periphery of the predetermined site,
wherein the medicine discharge unit selectively discharges at least one of the first medicine and the second medicine in accordance with a detection result obtained by the detection unit.

5. The medical device according to claim 1, further comprising:
a diagnosis unit configured to diagnose a condition of the periphery of the predetermined site when the medical device reaches the periphery of the predetermined site,
wherein the medicine discharge unit selectively discharges at least one of the first medicine and the second medicine in accordance with a diagnosis result obtained by the diagnosis unit.

6. The medical device according to claim 1, further comprising:
an imaging unit configured to image the intracorporeal site of the subject.

7. The medical device according to claim 1, comprising:
a sampling unit configured to sample a biological tissue from the intracorporeal site of the subject.

8. A medical system comprising:
a medical device including
a plurality of storage units each configured to store a different medicine, and
a medicine discharge unit configured to selectively discharge at least one of a first medicine and a second medicine, each of which is stored in the plurality of storage units; and
a control device including
a position detection unit configured to detect a position of the medical device, and
a control unit configured to perform control in a manner that a control signal for causing at least one of the first medicine and the second medicine to be discharged is transmitted to the medical device when the medical device is determined to have reached a periphery of a predetermined site of an intracorporeal site of a subject based on the position detected by the position detection unit.

9. The medical system according to claim 8, wherein, when medicine identification information included in the control signal transmitted from the control device is legitimate, the medicine discharge unit discharges at least one of the first medicine and the second medicine according to the control signal.

10. A program causing a computer to execute:
a process of selectively discharging at least one of a first medicine and a second medicine, each of which is stored in a plurality of storage units each storing a different medicine when a medical device reaches a periphery of a predetermined site of an intracorporeal site of a subject.
